(19) United States
(12) Patent Application Publication
(10) Pub. No.: US 2020/0282175 A1
(43) Pub. Date: Sep. 10, 2020

(54) Original Mind Gym

(71) Applicant: Daniel S. Vitchoff, Pittsburgh, PA (US)

(72) Inventor: Daniel S. Vitchoff, Pittsburgh, PA (US)

(73) Assignee: Daniel S. Vitchoff, Warrendale, PA (US)

(21) Appl. No.: 13/209,729

(22) Filed: Aug. 15, 2011

(51) Int. Cl. A61M 21/02

(52) U.S. Cl. CPC A61M 21/02; A61M 2021/0033

(57) ABSTRACT

The Original Mind Gym is a vital mental training system. It provides a physical and mental relaxation state combined with mental messaging using research based mental programming techniques. A Zero Gravity Chair, or any other suitable chair, combines physical comfort with powerful hypnotic suggestions, brain wave technology and soothing background music. The Original Mind Gym uses “The 33 Method™” to address multiple areas of personal development such as reducing stress and anxiety, building confidence, improving sports performance, weight loss, smoking cessation, personal and professional development. The Original mind gym has components that provide the capacity for real time face-to-face mental training at a distance.

The “33 Method™” mental training programs incorporate a unique combination of Sports Psychology, NLP (Neuro-linguistic Programming), Visualization and Imagery to help athletes achieve their maximum potential.

### BACKGROUND

### Field of the Disclosure

A research study which concentrated on brain focus as related to mental training, conducted at Waisman Laboratory for Brain Imaging and Behavior and Department of Psychology, University of Wisconsin, Madison concluded that “Meditation includes the mental training of attention, which involves the selection of goal-relevant information from the array of inputs that bombard our sensory systems. One of the major limitations of the attentional system concerns the ability to process two temporally close, task-relevant stimuli. When the second of two target stimuli is presented within a half second of the first one in a rapid sequence of events, it is often not detected. This so-called “attentional-blink” deficit is thought to result from competition between stimuli for limited attentional resources. We measured the effects of intense meditation on performance and scalp-recorded brain potentials in an attentional-blink task. We found that three months of intensive meditation reduced brain-resource allocation to the first target, enabling practitioners to more often detect the second target with no compromise in their ability to detect the first target. “These findings demonstrate that meditative training can improve performance on a novel task that requires the trained attentional abilities.”

http://www.plosbiology.org/article/info:doi/10.1371/journal.pbio.0050138

In addition, research published by the proceedings of the National Academy of Sciences of the United States of America as a result of an extensive scientific research study found in “meditation practice with the integrative body-mind training method shows significantly better attention and control of stress than similarly chosen control group given relaxation training.” These findings are significant as related to the Original Mind Gym because distracters were used, however not as sophisticated as the OMG distracters, the results of the study showed significantly better attention and control of stress.

http://www.ncbi.nlm.nih.gov/pmc/articles/PMC2040428/

The chair was designed by NASA engineers for the purpose of taking the stress off of the body of the astronauts during the lift off. Additional physical benefits of the chair are the lowering of blood pressure. As described by the developers of the chair this happens “by allowing you to angle your lower legs above the level of your heart. This increases blood flow around the body, places less stress on your heart and therefore your blood pressure is lowered.” The design of the chair also takes the pressure off of the spine thus engaging the body in additional relaxation state.

The benefits that the chair provides, enhances the benefit of the hypnotic and meditative processes by relaxing the body thus beginning the process of relaxing the mind. The induction process of meditation and hypnosis is the awareness of the body for the purpose of relaxation and preparation of the mind.

The Original Mind Gym is a multi-level, multi-sensory, program that complements the each affect needed to achieve an intense experience of relaxation both physically and mentally. The Hypnotic suggestions are welcomed through a relaxed body and mind that is focused completely on the goal to be achieved through the subconscious mind.

http://ezinearticles.com/?Zero-Gravity-Chair-Benefits&id=2617002

Article Source: http://EzineArticles.com/2617002

### Background of the Disclosure

The initial quest to help individuals who struggle with making positive changes in their lives was the beginning of the OMG. As I continued to work with various individuals with varied needs, I found that the combination of the techniques and processes that I was using were very successful in helping people change their lives, and be successful meeting personal and professional goals. The impetus of the OMG grew after the 2008 Olympic games. I incorporated the OMG into the mental training that I conducted for the USAMU as they prepared for the 2008 games. Two of the competitors won a gold medal. Likewise, the use of the techniques helped hundreds of my clients in their quest to overcome anxiety, lose weight, quit smoking and conquer dealing with PTSD.

Stress manifests its self in many ways. It invades the body and the mind. In my opinion, there is a need for the OMG because it is an avenue to help people live a more healthy and productive life. The value of a mentally and physically healthy person cannot be underestimated for the individual or for the society as a whole.

### BRIEF SUMMARY OF INVENTION

The Original Mind Gym offers the user physical and mental comfort and relaxation while experiencing individualized mental training exercises via the various components of the invention. A Zero Gravity Chair, or any other suitable chair, combining physical comfort with powerful hypnotic suggestions, brain wave technology and soothing background music which comfort and relax the mind. The Original Mind Gym uses “The 33 Method™” to address multiple areas of personal development such as reducing stress and anxiety, building confidence, improving sports performance, weight loss, smoking cessation, personal and professional development. The capacity for real time face-to-face mental training sessions are built in to the system.

The “33 Method™” mental training programs incorporate a unique combination of Sports Psychology, NLP (Neuro-linguistic Programming), Visualization and Imagery to help athletes achieve their maximum potential.

Research Behind the OMG

The chair was designed by NASA engineers for the purpose of taking the stress off of the body of the astronauts during the lift off. Additional physical benefits of the chair are the lowering of blood pressure. As described by the developers of the chair this happens “by allowing you to angle your lower legs above the level of your heart.

This increases blood flow around the body, places less stress on your heart and therefore your blood pressure is lowered.” The design of the chair also takes the pressure off of the spine thus engaging the body in additional relaxation state. The OMG uses this aspect of relaxation as a segment in the preparation of body to free the mind for attention to mental training of the user.

A research study which focused on brain focus as related to mental training conducted at Waisman Laboratory for Brain Imaging and Behavior and Department of Psychology, University of Wisconsin, Madison concluded that “Meditation includes the mental training of attention, which involves the selection of goal-relevant information from the array of inputs that bombard our sensory systems.

The Original Mind Gym is the ultimate relaxation experience. A Zero Gravity Chair, or any other suitable chair, combines physical comfort with powerful hypnotic suggestions, brain wave technology and soothing background music. The Original Mind Gym uses “The 33 Method™” to address multiple areas of personal development such as reducing stress and anxiety, building confidence, improving sports performance, weight loss, smoking cessation, personal and professional development.

### BRIEF DESCRIPTION OF THE DRAWINGS

Selected embodiments of the disclosure may be understood, and its numerous objects and features obtained, when the following detailed description is considered in conjunction with the following drawings, in which:

FIG. 1 depicts the Zero Gravity Chair in which a user would sit to gain an ultimate relaxation experience.

FIG. 2 depicts the attachment that connects the touch screen device to the Zero Gravity Chair.

FIG. 3 depicts the touch screen device that a user will interact with to launch different mental training exercises.

FIG. 4 depicts the Zero Gravity Chair as it is attached to the Touch Screen Device.

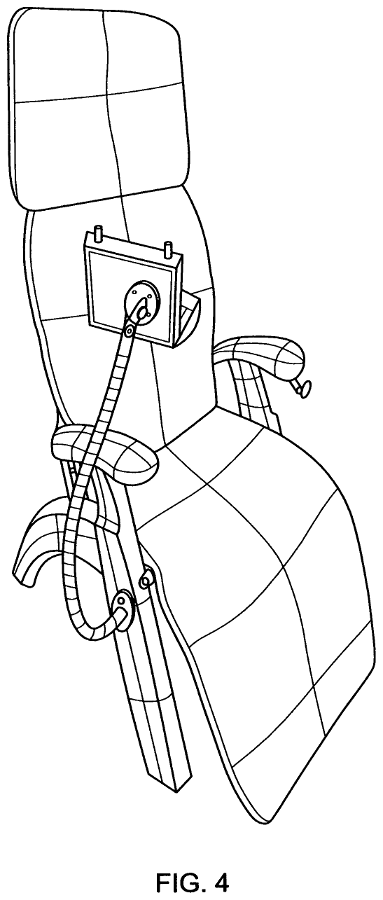

### DETAILED DESCRIPTION

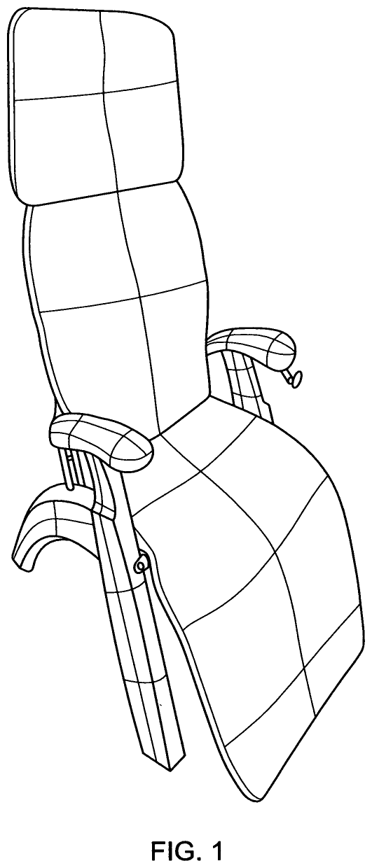

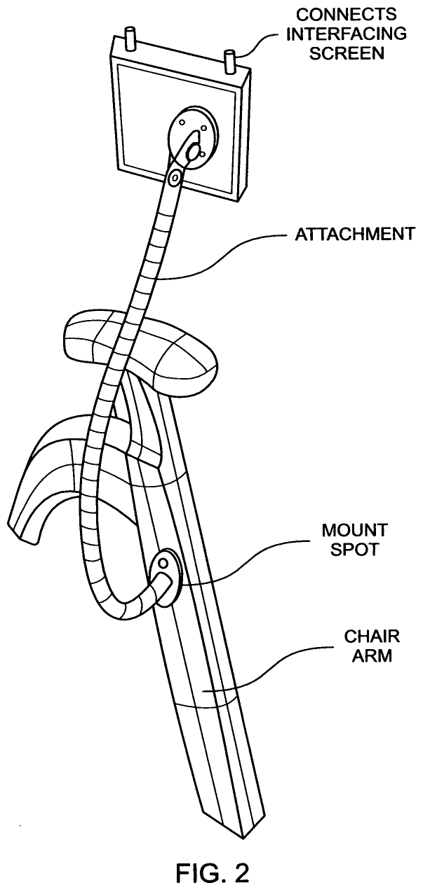

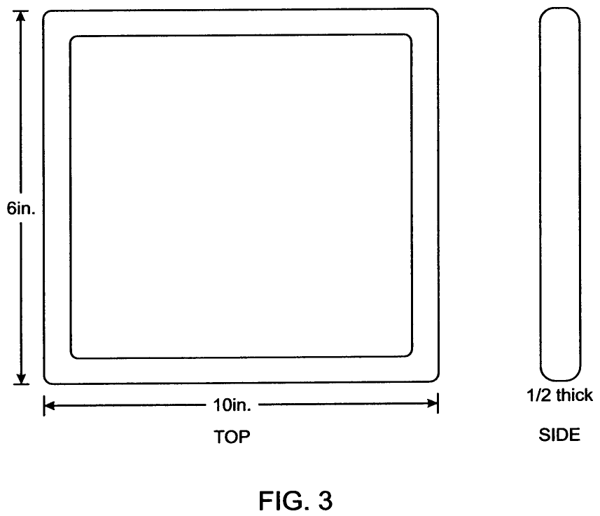

The Original Mind Gym can be described as the ultimate relaxation experience. The Original Mind Gym promotes physical and emotional comfort and relaxation by stimulating three of the five senses; sight, sound and touch. The Original Mind Gym is made up of 6 key components:
1. Zero Gravity Chair (FIG. 1)
2. Attachment that connects the Touch Screen Device to the Zero Gravity Chair. (FIG. 2)
3. A Touch Screen Control Device with the capability of playing Hypnosis and Mental Training Sessions. The touch screen device is equipped with a headphone jack. (FIG. 3)
4. Noise Cancelling Headphones
5. Glasses equipped with brain wave technology where light impulses will stimulate the optic nerve.
6. Audio Capabilities to play hypnosis/mental training sessions.

The Zero Gravity Chair (FIG. 1) offers the user the ability to recline to a body position very similar to a lying down position in which their feet are elevated above the level of their heart which increases blood flow and oxygen to the brain. This body position naturally allows the body to feel light and weightless and release physical stress and tension.

Their origin dates back to NASA when the astronauts needed to recline their body position in such a way their legs are higher than their heart level during a takeoff. The user's body weight is evenly distributed across their entire body. Even chiropractors (spine specialists) and physiologists support the use of these chairs for its many health advantages. A zero gravity chair is also known as a zero gravity recliner or anti gravity chair.

The attachment (FIG. 2) connects the touch screen device to the zero gravity chair. This attachment is sturdy yet flexible so that it can hold the weight of the touch screen device and still enable the user to position the touch screen to suit their level of comfort and ease of use.

The touch screen device (FIG. 3) has audio capabilities to play hypnosis/mental training sessions. The device will be preloaded with the appropriate files for each user and will also have the capability of being updated by the administrator from a remote location. The device is equipped with a SD Card Slot and headphone jack. The 9″ Touch Screen Tablet merges the power of a desktop and the portability of a netbook with the precision of cutting edge portable multimedia. The thin and sleek slate pc displays a 9″ wide, matte-finish touch screen with High Definition video. With Windows 7 and a 60 GB hard drive enables us to install our own customized software. All of these features come in one 29 oz device. The ergonomic shape is only 0.67 inches thick which makes it aesthetically pleasing when attached to the Zero Gravity Chair.

Additional features include a built-in webcam for skype and virtual face to face coaching and consulting, Wi-Fi, stereo speakers, and a microphone to use with your favorite video chat service from anywhere there is a wireless internet connection.

The user will use noise cancelling headphones to eliminate external noises and distractions. Noise cancelling headphones hush ambient noise by creating anti-noise that obviates the noise at your ear. They don't eliminate noise, but they significantly reduce the distraction of common background noises that may exist in locker rooms, training facilities and day spas such as talking, telephones ringing and doors opening and closing.

The light and sound glasses that are equipped with brain wave technology in which impulses of lights stimulate the optic nerve.

The Original Mind Gym is the ultimate relaxation experience. A Zero Gravity Chair, or any other suitable chair, combines physical comfort with powerful hypnotic suggestions, brain wave technology and soothing background music. The Original Mind Gym uses “The 33 Method™” to address multiple areas of personal development such as reducing stress and anxiety, building confidence, improving sports performance, weight loss, smoking cessation, personal and professional development.

The Original Mind Gym offers the user physical and mental comfort and relaxation. A Zero Gravity Chair, or any other suitable chair, combines physical comfort with powerful hypnotic suggestions, brain wave technology and soothing background music which comfort and relax the mind. The Original Mind Gym uses “The 33 Method™” to address multiple areas of personal development such as reducing stress and anxiety, building confidence, improving sports performance, weight loss, smoking cessation, personal and professional development.

The OMG integrates all elements known to be effective in sports psychology, relaxation, mental training, and behavior psychology. Cognitive and behavior techniques are encompassed in the software while the physical effects are attended to by the chair.

1. A system for self-sustained hypnosis, the system comprising:
a chair;
headphones;
glasses;
and a touch screen device;
where in the headphones, glasses and touch screen device are attached to the chair.

2. The system of claim 1, wherein the headphones are noise-cancelling headphones.

3. The system of claim 1, wherein the chair is a zero-gravity chair or any chair that takes pressure off of the spine, positions the body at an angle that lowers blood pressure and creates a sensation of relaxation, and weightlessness.

4. The system of claim 1, wherein the glasses provide a variation of a series of light flashes adjusted to the user's preferred series or clinician's recommended series.

5. The system of claim 1, wherein the touch device delivers hypnosis, neuro-linguistic programming (NLP) and behavior modification hypnotic suggestions.

6. A method for self-sustained hypnosis, the method comprising:
a chair;
headphones;
glasses; and
a touch screen device;
wherein the headphones, glasses and touch screen device are attached to the chair, wherein the chair initiates physical relaxation via: 1) placing the body at an angle where the legs are above the heart thus lowering of blood pressure which enables the body to relax more easily with the blood pressure lowered and less stress on the heart, and 2) the chair also takes the pressure off of the spine thus engaging the body in additional relaxation state.

7. The method of claim 6, wherein the headphones cancel outside noise and the following is heard
1. an induction,
2. a series of proprietary behavioral modification, NLP and hypnotic suggestions and,
3. synchronized background music.

8. The method of claim 7, wherein the glasses provide a variation of a series of light flashes adjusted to the users preferred series and intensity.

9. The method of claim 6, wherein the touch device delivers
1. an Induction,
2. a series of hypnosis, proprietary behavioral modification, cognitive exercises, NLP and hypnotic suggestions and,
3. synchronized background music and,
4. conclusion through the Awakening.

10. An apparatus for self-sustained hypnosis, comprising:
a chair;
headphones;
glasses;
and a touch screen device.

11. The apparatus of claim 10 further comprising a reclining mechanism on the chair that adjusts to various angles.

12. The apparatus of claim 10 further comprising noise cancelling headphones.

13. The apparatus of claim 10 further comprising glasses that execute a session of light sequences.

14. The apparatus of claim 10 further comprising a touch screen device that houses software.
